(19) United States
(12) Patent Application Publication
(10) Pub. No.: US 2007/0066917 A1
(43) Pub. Date: Mar. 22, 2007

(54) Method for simulating prosthetic implant selection and placement

(76) Inventors: Robert A. Hodorek, Warsaw, IN (US); James E. Grimm, Winona Lake, IN (US)

Correspondence Address: ZIMMER TECHNOLOGY - BAKER & DANIELS, 111 EAST WAYNE STREET, SUITE 800, FORT WAYNE, IN, 46802, US

(21) Appl. No.: 11/231,156

(22) Filed: Sep. 20, 2005

(51) Int. Cl. A61B 5/11

(52) U.S. Cl. 600/595

(57) ABSTRACT

A method and apparatus are provided for preoperatively or intraoperatively determining prosthetic implant selection and placement to achieve acceptable alignment and spacing of anatomical structures affected by the prosthetic implant and to achieve acceptable soft tissue balance proximate the prosthetic implant without requiring trial-and-error selection of implant size and placement during surgery. In one exemplary embodiment, the method and apparatus of the present invention are used to choose appropriate tibial, meniscal and femoral prosthetic components to achieve acceptable alignment, acceptable spacing of the tibia and femur, and acceptable soft tissue balance over a full range of motion of the knee.

### BACKGROUND

1. Field of the Invention

The present invention relates to computer-assisted surgery and, more specifically, to a method and apparatus for simulating prosthetic implant selection and placement using a computer-assisted surgery system.

2. Description of the Prior Art

Computer-assisted surgical systems and procedures have been developed for positioning surgical instruments in a predefined position and orientation relative to a patient's anatomical structures. Computer-assisted guidance of surgical instruments can be used in orthopedic surgical procedures to, e.g., position a cutting instrument in a predefined position and orientation with respect to a bone when preparing the bone to receive a prosthetic implant such as a component of an artificial joint. Guidance techniques typically involve acquiring preoperative images of the relevant anatomical structures and generating a database which represents a three-dimensional model of the anatomical structures. The relevant surgical instruments typically have a fixed geometry which is used to create geometric models of the instruments. The geometric models of the relevant instruments can be superimposed on the model of the relevant anatomical structures.

During the surgical procedure, the position of the instrument(s) being used and the patient's anatomical structures are registered with the anatomical coordinate system of the computer model of the relevant anatomical structures. Registration is the process of defining the geometric relationship between the physical world and a computer model. Registration of the patient with the computer model allows the computer to manipulate the computer model to match the relative positions of various components of the patient's anatomical structure in the physical world. Registration of the instrument(s) used with the computer model allows the computer to display and/or direct the placement of the instrument(s) and prosthetic components relative to the patient's anatomical structure. To assist the registration process, pins or markers are placed in contact with a portion of the anatomical structure which are also locatable in the computer model. The markers are locatable in space by the computer, thereby providing a geometric relationship between the model and physical anatomical structure. A graphical display showing the relative positions of the instrument and anatomical structures can then be computed in real time and displayed to assist the surgeon in properly positioning and manipulating the surgical instrument with respect to the relevant anatomical structure. Examples of various computer-assisted navigation systems are described in U.S. Pat. Nos. 5,682,886; 5,921,992; 6,096,050; 6,348,058; 6,434,507; 6,450,978; 6,470,207; 6,490,467; and 6,491,699, the disclosures of which are hereby explicitly incorporated herein by reference.

In traditional knee arthroplasty, achieving proper limb alignment and proper soft tissue balance requires a trial-and-error technique. In this trial-and-error technique, the surgeon generally makes one of the distal femoral cut and the proximal tibial cut, and thereafter selects the location of the other of the distal femoral cut and the proximal tibial cut based on experience and the knowledge that tibial prosthetic implants are available in a limited number of thicknesses. The remaining femoral cuts are made to complete shaping of the femur to receive a femoral prosthesis. After the femoral and tibial cuts are complete, the femoral prosthesis and the tibial prosthesis, or provisional versions thereof, are temporarily implanted and leg alignment and soft tissue tension are examined by the surgeon.

To adjust leg alignment or soft tissue tension, the surgeon can, e.g., replace the tibial prosthesis or a meniscal component of the prosthesis with alternative components having increased or decreased thicknesses and/or recut the tibia. The surgeon may also recut the femur and/or use a different femoral implant to achieve appropriate leg alignment and soft tissue tension. The surgeon can also perform ligament releases or advances to adjust and balance soft tissue tension. Changes in implant component choice and location are made and soft tissue balance is rechecked in a trial-and-error procedure until the surgeon is satisfied with leg alignment and soft tissue balance.

### SUMMARY

A method and apparatus are provided for preoperatively or intraoperatively determining prosthetic implant selection and placement to achieve acceptable alignment and spacing of anatomical structures affected by the prosthetic implant and to achieve acceptable soft tissue balance proximate the prosthetic implant without requiring trial-and-error selection of implant size and placement during surgery. In one exemplary embodiment, the method and apparatus of the present invention may be used in prosthetic knee surgery to choose appropriate tibial, meniscal and femoral prosthetic components to achieve acceptable alignment, acceptable spacing of the tibia and femur, and acceptable soft tissue balance over a full range of motion of the knee.

In one form thereof, the present invention provides a method for simulating prosthetic implant selection and placement in an anatomical structure using a computer-assisted surgery system, including the steps of generating a virtual model of the anatomical structure; registering the anatomical structure with the virtual model of the anatomical structure in the computer-assisted surgery system; determining a mechanical axis correction of the anatomical structure; determining soft tissue balance in the anatomical structure; selecting a simulated implant component corresponding to the mechanical axis correction and the soft tissue balance; simulating implantation of the simulated implant component; verifying that the simulated implant component provides the mechanical axis correction and the soft tissue balance; and selecting an actual implant component corresponding to the simulated implant component if the simulated implant component provides the mechanical axis correction and the soft tissue balance.

In another form thereof, the present invention provides a method for simulating prosthetic implant selection and placement in a knee joint using a computer-assisted surgery system, the knee joint including a femur and a tibia, including the steps of generating a virtual model of the knee joint; registering the knee joint with the virtual model of the knee joint in the computer-assisted surgery system; determining a mechanical axis correction of the knee joint; determining soft tissue balance in the knee joint; selecting a simulated implant component corresponding to the mechanical axis correction and the soft tissue balance; simulating implantation of the simulated implant component; verifying that the simulated implant component provides the mechanical axis correction and the soft tissue balance; and selecting an actual implant component corresponding to the simulated implant component if the simulated implant component provides the mechanical axis correction and the soft tissue balance.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is a perspective view of an operating room arrangement including a computer-assisted surgical system according to an embodiment of the present invention, and further showing a patient;

FIG. 2 is a plan view of a first graphical display of the computer-assisted surgical system of FIG. 1, the display providing graphical information and data regarding patient anatomical structures and prosthetic implant components;

FIG. 3 is an anterior/posterior view of a femur and tibia showing a corrected mechanical axis;

FIG. 4A is an anterior/posterior view of a knee joint;

FIG. 4B is a lateral view of a limb including the knee joint of FIG. 4A;

FIG. 5 is a plan view of a second graphical display of the computer-assisted surgery system of FIG. 1, the display showing simulated placement of a femoral and tibial implant in extension;

FIG. 6 is a plan view of a third graphical display of the computer-assisted surgery system of FIG. 1, the display showing simulated placement of a femoral and tibial implant in 90° flexion;

FIG. 7 is a perspective view of a surgical instrument and a computer navigation device of the computer-assisted surgery system of FIG. 1 used to perform a proximal tibial cut in accordance with the present invention;

FIGS. 8A and 8B are a flow chart illustrating a method for determining prosthetic implant selection and placement using a computer-assisted navigation system according to the present invention; and

FIG. 9 is a block schematic diagram of the computer-assisted surgery system of FIG. 1.

Corresponding reference characters indicate corresponding parts throughout the several views. Although the drawings represent embodiments of the present invention, the drawings are not necessarily to scale and certain features may be exaggerated to better illustrate and explain the present invention. The exemplifications set out herein illustrate embodiments of the invention, in several forms, and such exemplifications are not to be construed as limiting the scope of the invention in any manner.

### DETAILED DESCRIPTION

The embodiments disclosed below are not intended to be exhaustive or limit the invention to the precise forms disclosed in the following detailed description. Rather, the embodiments are chosen and described so that others skilled in the art may utilize their teachings. While the description that follows refers to implantation of a prosthetic knee, the teachings of the present invention are readily adaptable to implantation of any prosthesis, including a prosthesis for partial or complete replacement of the hip, shoulder, wrist, elbow, or ankle.

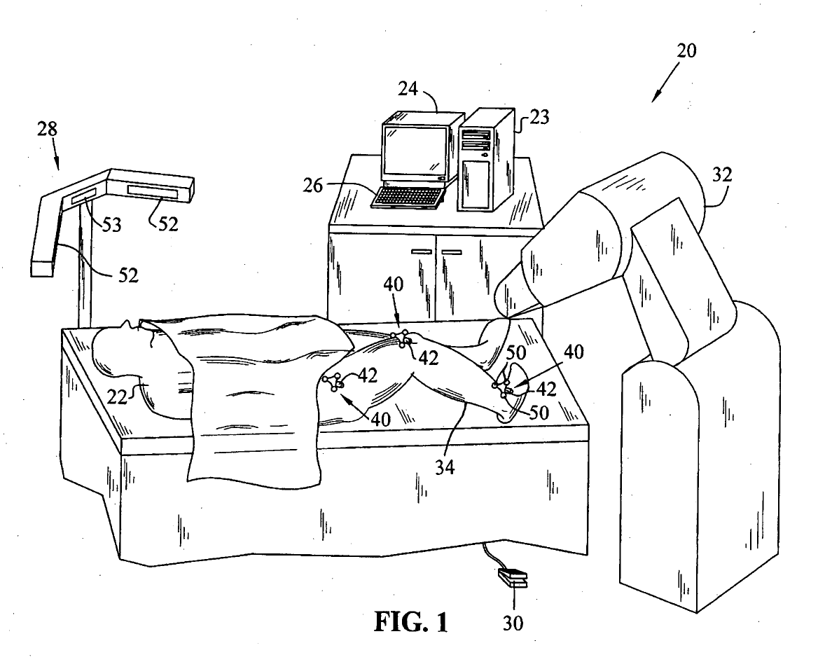

FIG. 1 shows an operating room arrangement having computer-assisted surgery system 20 for aiding surgical procedures performed on patient 22. As described herein, computer-assisted surgery system 20 may be used to provide graphical and other data information relating to the anatomical structures of patient 22 and to simulate prosthetic implant selection and placement preoperatively or intraoperatively to minimize or eliminate in vivo trial-and-error surgical procedures for positioning a prosthesis.

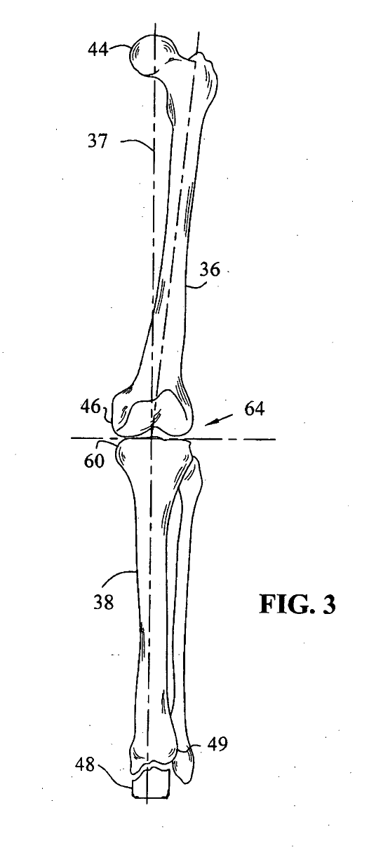

Referring still to FIG. 1, system 20 may include computer 23, display 24, keyboard 26, navigation sensor 28, input device 30 and imaging device 32. Generally, computer 23 and navigation sensor 28 determine the position of anatomical structures of patient 22, for example, the position of limb 34 including femur 36 and tibia 38 (FIG. 3) may be determined. Navigation sensor 28 detects the position of the anatomical structures by sensing the position and orientation of markers such as reference arrays 40 associated with the anatomical structures. Each reference array 40 may include probe 42 extending through an incision in limb 34 and contacting a bone landmark, for example femoral head 44, distal femur 46, and/or talus 48 (FIG. 3). Each reference array 40 includes an array of reference devices 50 which passively or actively transmit an optical, electromagnetic, or other signal to sensors 52 of navigation sensor 28. If a passive reference device 50 is used, emitter 53 transmits a signal that is reflected by reference device 50 and then received by sensors 52 upon reflection from reference device 50. If an active reference device 50 is utilized, reference device 50 itself generates a signal for transmission to, and detection by, sensors 52.

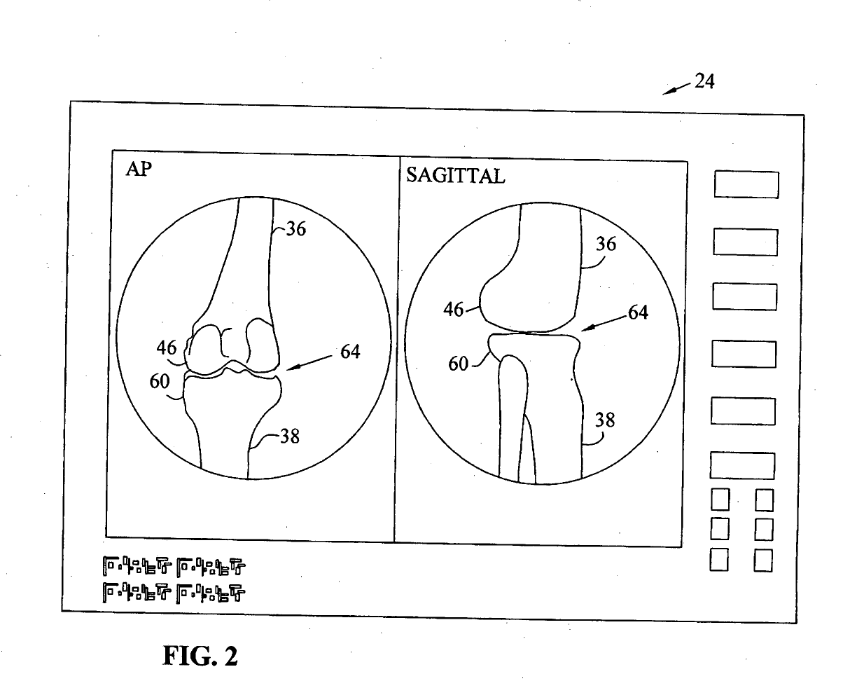

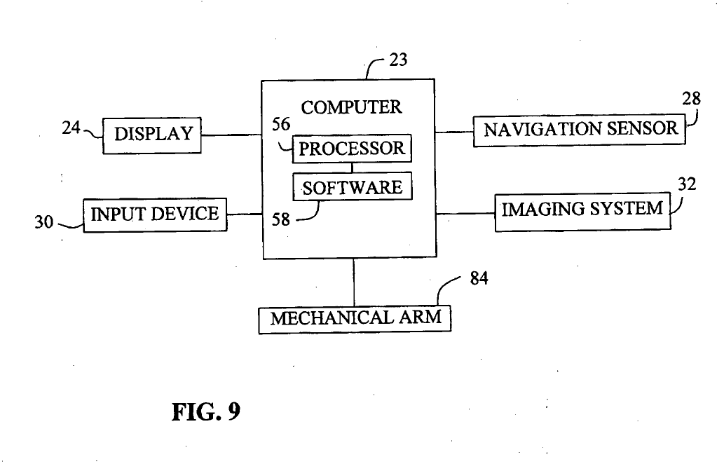

Computer 23, shown in FIGS. 1 and 9, includes processor 56 and software 58. Software 58 provides tracking of reference arrays 40 so that graphical and data representations of the anatomical structures of patient 22 may be provided on display 24. As illustrated in FIG. 2, representations of knee joint 64 may be shown on display 24, for example. While not illustrated in FIG. 2, the ligaments surrounding knee joint 64 may also be imaged and modeled together with the bones of knee joint 64. To enhance the displayed image and to provide a three-dimensional model of the anatomical structures, imaging device 32 may be used for providing images of the anatomical structures to computer 23. Imaging device 32 may be any of the well-known devices utilized for providing images of internal body structures, such as a fluoroscopic imaging device, a computerized tomography (CT) imaging device, a magnetic resonance imaging (MRI) device, an ultrasound imaging device, or a diffraction enhanced imaging (DEI) device.

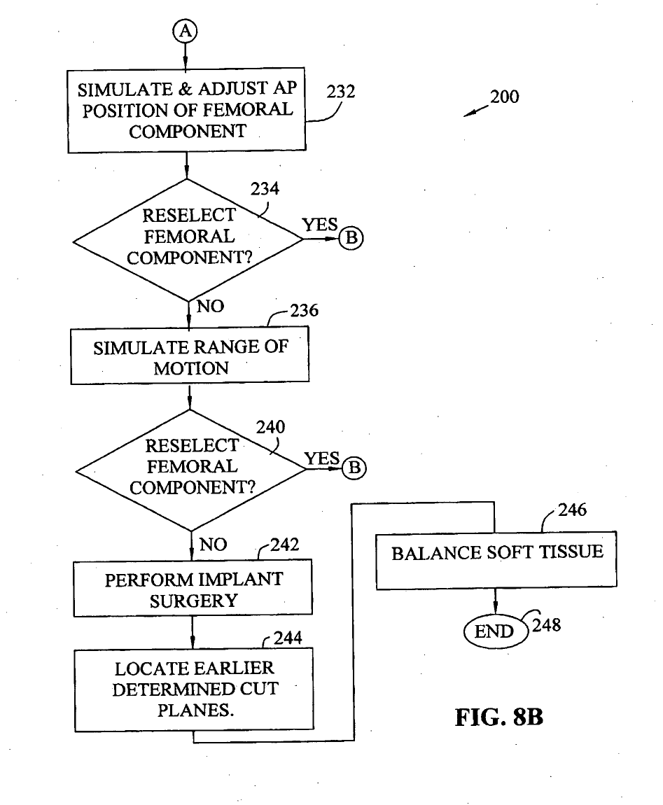

The following description of an exemplary method of the present invention is directed to a total knee arthroplasty. As previously indicated, however, the method and apparatus of the present invention are usable with the placement of any prosthesis. Referring to FIGS. 8A and 8B, method 200 includes steps that, at least in part, may be implemented by software 58 and other components of computer-assisted surgery system 20. Certain steps may also require activity from a surgeon or other person. Method 200 begins at step 202 and may be performed preoperatively or intraoperatively.

In step 204, reference arrays 40 (FIG. 1) are located at various bone landmarks of limb 34 (FIG. 1), for example and as shown in FIG. 3, femoral head 44, distal femur 46, talus 48 and/or distal tibia 49 may be located and marked by reference arrays 40. As described previously and referring to FIG. 1, reference arrays 40 may include reference devices 50 which are tracked by navigation sensor 28. Reference array 40 may also include probe 42 which extends through an incision in limb 34 and contacts the desired bone landmarks. Alternatively, the bone landmarks may be located by reference devices 50 which do not penetrate limb 34 and are positioned securely relative to limb 34 by other surgical instrumentation.

In step 206, imaging device 32 (FIG. 1) is used to provide images of the anatomical structures to computer 23. In one embodiment, multiple fluoroscopic images may be used to construct three-dimensional images of the appropriate anatomical structures. Alternatively, images from CT imaging devices, a combination of fluoroscopic and CT imaging devices, MRI devices, ultrasound imaging devices, or DEI devices, may be used. The soft tissues of the knee, including the surrounding ligaments may also be imaged during step 206 and added to the virtual model of the knee. Referring to FIG. 2, pre-implant display is shown on display 24 having exemplary anterior/posterior (hereinafter “AP”) and sagittal plane views of distal femur 46 and proximal tibia 60. Alternatively, other views, including views illustrating the relevant soft tissues surrounding knee joint 64 may be utilized.

In step 208, the relevant anatomical structures are registered with computer-assisted surgery system 20. Specifically, the combination of data available from reference devices 50 and images of the anatomical structures form a model of knee joint 64 seen in the pre-implant display of FIG. 2.

The model may be further developed by specifying additional landmarks of the anatomical structures which are visible in the AP and sagittal plane views of the pre-implant display of FIG. 2. The resulting three-dimensional model and images may be overlaid together and used to provide accurate display and simulation of the anatomical structures, including mechanical axis 37 (FIG. 3) which extends from femoral head 44, through the center of distal femur 46, proximal tibia 60 and distal tibia 49. As previously indicated, the soft tissues surrounding knee joint 64, including the surrounding ligaments, may form a part of this display. In one exemplary embodiment (not shown), a pair of models of knee joint 64, one including soft tissue and one not including soft tissue are generated and displayed simultaneously.

In step 210, the surgeon may determine the desired correction for mechanical axis 37 to correct for varus and valgus defects. The surgeon may hold limb 34 in extension, as shown in FIGS. 2, 3, and 4A, manually or with the assistance of instrumentation, and manipulate limb 34 such that the anatomical structures cooperate to form a satisfactory and correct mechanical axis 37. An example of such correction is described in “Method and Apparatus for Achieving Correct Limb Alignment in Unicondylar Knee Arthroplasty,” U.S. patent application Ser. No. 10/305,697, filed on Nov. 27, 2002, assigned to the assignee of the present invention, the disclosure of which is hereby explicitly incorporated herein by reference.

AP and sagittal plane views of the pre-implant display of FIG. 2 may provide guidance information for correcting a varus or valgus deformity. When the surgeon has placed limb 34 in the correct position, input device 30 (FIG. 1) may be actuated to store the desired mechanical axis correction, i.e., the relative positions of femur 36 and tibia 38 forming a satisfactory mechanical axis 37. In one embodiment, as shown in FIG. 1, input device 30 may be a foot-operated actuator used to capture images of the relative position of the anatomical structures during manipulation of knee joint 64 by a surgeon.

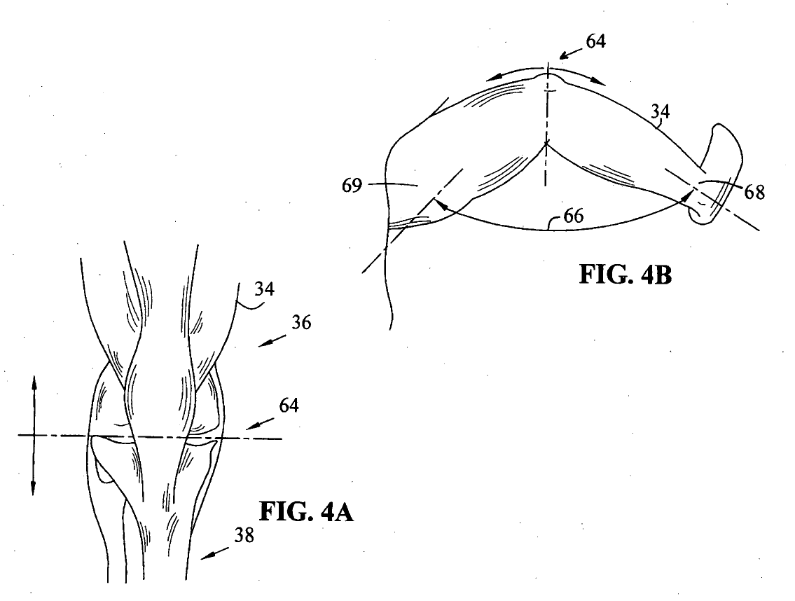

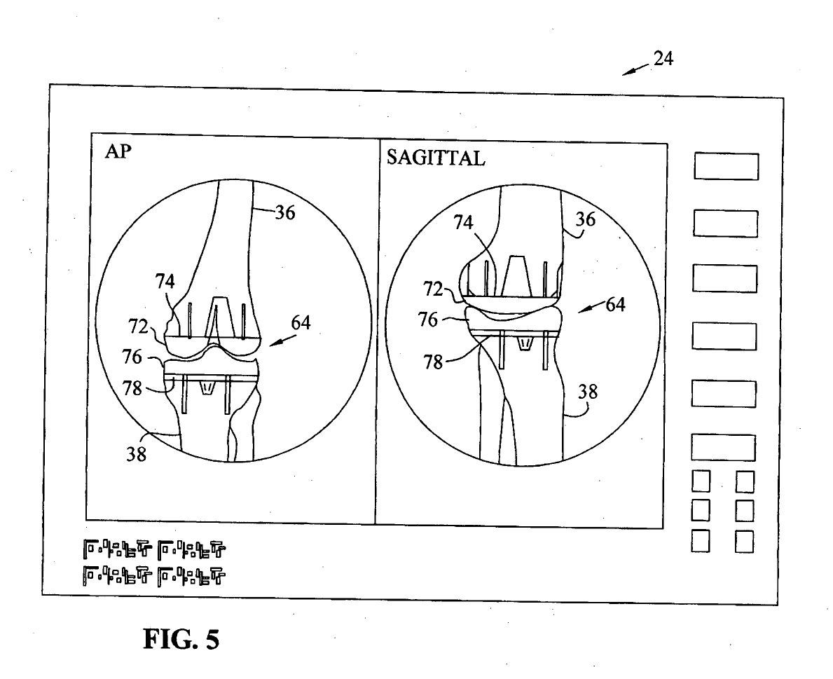

In step 212, the soft tissue balance around knee joint 64 is evaluated in extension and a desired balance may be specified and stored by computer 23. Referring to FIG. 4A, soft tissue laxity, or lack of tension, in the soft tissues proximate knee joint 64, such as the collaterals, capsules, posterior cruciate ligament (hereinafter “PCL”), and anterior cruciate ligament (hereinafter “ACL”), is often excessive in patients requiring knee arthroscopy. Therefore, laxity is often reduced during the prosthetic implantation process either before or after the implant components are positioned. The surgeon may hold limb 34 and displace tibia 38 away from knee joint 64 until the desired amount of tension is achieved. When the desired tension is achieved, input device 30 (FIG. 1) may be actuated to store the relationship in extension between tibia 38 and femur 36 that is required to provide the desired level of soft tissue balance. In one embodiment, the surgeon may displace tibia 38 by manually pulling tibia 38 away from knee joint 64. Alternatively, the surgeon may use a laminar spreader or other tensioning device (not shown), including, for example, a tension gauge which may be coupled to computer 23, to displace tibia 38 away from knee joint 64.

In step 214, the soft tissue balance around knee joint 64 is evaluated in flexion and a desired balance may be specified and stored by computer 23. Referring to FIG. 4B, a surgeon may flex limb 34 to a desired flexion angle 66 between ankle 68 and hip 69, for example, 90°, or, alternatively, 1450 for deep flexion. At the desired flexion angle 66, the soft tissue balance may be evaluated and limb 34 positioned similarly to positioning limb 34 in extension, as described above, to achieve a relationship between the anatomical structures which provides the desired soft tissue balance in flexion. When the desired tension is achieved, input device 30 (FIG. 1) may be actuated to store the relationship in flexion between tibia 38 and femur 36 that is required to provide the desired level of soft tissue balance.

In addition to storing the relationship in extension and flexion between tibia 38 and femur 36 that is required to provide the desired level of soft tissue balance, the surgeon may move limb 34 through a series of positions between extension and 90° flexion, as well as beyond 90° flexion, and at each position store the relationship between tibia 38 and femur 36 that is required to provide the desired level of soft tissue balance. In this manner, the surgeon can store the relationship between tibia 38 and femur 36 that is required to provide the desired level of soft tissue balance throughout the entire range of motion of knee joint 64.

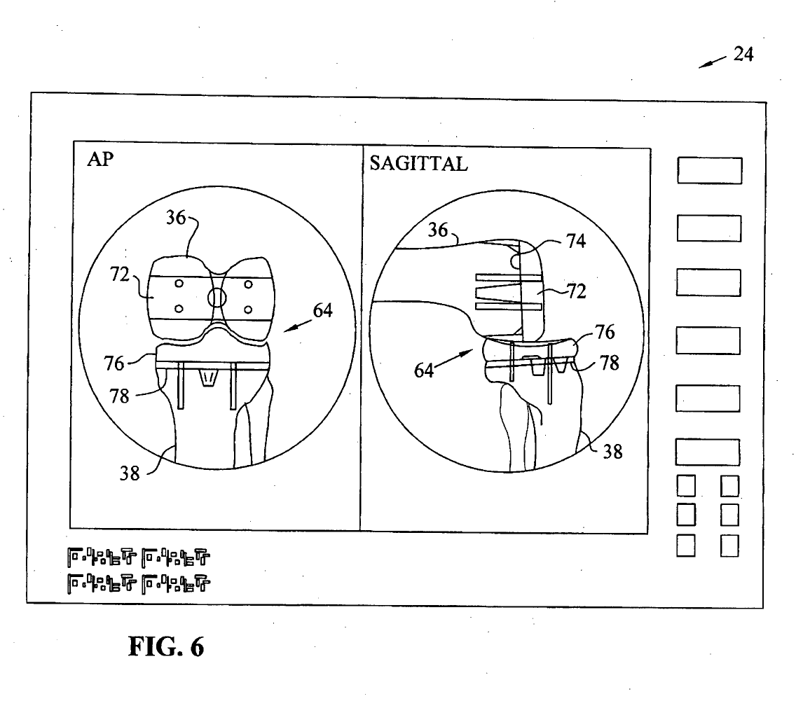

Referring now to FIG. 5, in step 216, a simulated femoral implant component 72 is selected and shown implanted in simulated extended knee joint 64. The selection of the simulated femoral implant component 72 may be manually done via keyboard 26 by the surgeon or selected by computer 23 based on the desired soft tissue balance and mechanical axis correction. Computer 23 may select simulated femoral implant component 72 based on software containing logic that uses information provided to computer 23 for the correct mechanical axis and soft tissue balance for the individual patient. Computer 23 selects femoral implant component 72 based on the anterior/posterior dimension matching the bone as well as filling the joint space in flexion. Once the femoral size is chosen, femoral implant component 72 can be optimally positioned on femur 36 (in both the proximal/distal as well as the anterior/posterior directions) to accommodate the gap provided by the soft tissue throughout the range of motion. Distal femoral cut plane 74 is located based on the three-dimensional model and image of the anatomical structures and the desired soft tissue balance and mechanical axis correction which are stored by computer 23. In step 218, placement of initial femoral implant component 72 relative to distal femoral cut plane 74 is simulated by computer 23 and a graphical model of femoral implant component 72 and the anatomical structures of knee joint 64 are displayed in the extension view shown in FIG. 5. Although the steps of locating distal femoral cut plane,74 and simulating the placement of femoral implant component 72 are typically completed before the steps of locating proximal tibial cut plane 78 (described below) and simulating placement of tibial implant component 76, for purposes of illustration, FIG. 5 shows an exemplary graphical display of both tibial implant component 76 and femoral implant component 72 seated in tibia 38 and femur 36, respectively.

Referring still to FIG. 5, in step 220, the surgeon may view the sagittal plane view of knee joint 64 in extension and may toggle through a pre-stored library of femoral component models in order to replace initial femoral implant component 72 with a different femoral implant component 72 based on anterior to posterior sizing, if necessary. In step 222, computer 23 simulates the desired relative positions of femur 36 and tibia 38 in extension to facilitate simulated selection and placement of tibial implant component 76. The initial selection of initial tibial implant component 76 may be done in a similar manner as that used to select initial femoral implant component 72, as described above. In step 224, the surgeon may use computer 23 to virtually determine proximal tibial cut plane 78 which will provide the appropriate contact of tibial implant component 76 with femoral implant component 72. In step 226, computer 23 simulates placement of tibial implant component 76 with the three-dimensional model and image of the anatomical structures, as shown in FIG. 5. In step 228, software 58 determines the position of the remaining femoral cuts required to position femoral implant component 72.

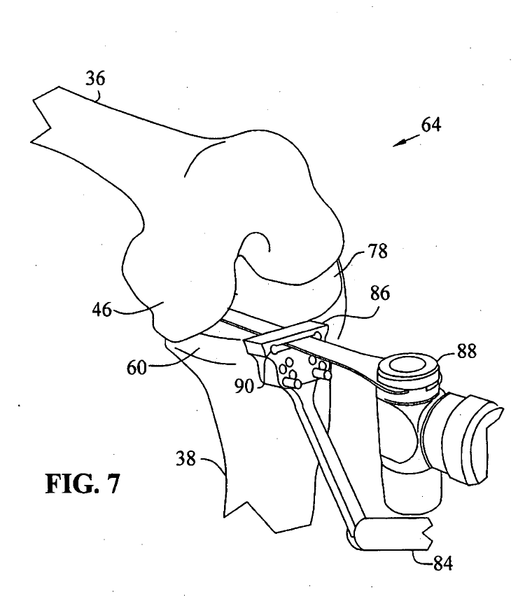

In step 230, computer 23 simulates 90° flexion of the anatomical structures of knee joint 64 and displays the flexion view on display 24, as shown in FIG. 6. Although the displayed flexion angle in FIG. 6 is 90°, another or multiple flexion angles may be simulated to predict the soft tissue balance and compare it with the desired soft tissue balance for the simulated flexion angle. Generally, it is desirable for knee joint 64 to have the same soft tissue balance for extension and 90° flexion, as well as every position therebetween. Referring to FIG. 8B, in step 232, computer 23 simulates the AP position of femoral implant component 72. The surgeon may then virtually adjust the AP position in order to provide the desired contact with tibial implant component 76 through a full range of motion from extension through flexion.

In step 234, the surgeon decides whether simulated reselection of femoral implant component 72 is necessary in order to adjust the predicted gap between femoral implant component 72 and tibial implant component 76 to provide the desired soft tissue balance. If reselection is desired, method 200 returns to step 220 (FIG. 8A). Alternatively, if reselection is not desired, step 236 is completed. In step 236, the geometric models of the anatomical structures of knee joint 64 and the chosen prosthetic components are used to perform biomechanical simulations of a full range of motion of knee joint 64. In this way, the alignment and spacing of femur 36 and tibia 38, as well as soft tissue balance, can be virtually evaluated before actual implantation of the prosthetic components or actual cutting of femur 36 and tibia 38. In step 240, the surgeon determines whether reselection or repositioning of femoral implant component 72, or tibial implant component 76, in computer 23 is required in order to correct mechanical axis 37 or the predicted soft tissue balance. If reselection or repositioning is required, the method returns to step 220. Alternatively, if reselection or repositioning is not required, the method moves on to step 242.

In step 242, the simulation is complete and actual implant surgery may be performed accordingly to the simulated plan/selection of femoral implant component 72, distal femoral cut plane 74, tibial implant component 76, and proximal tibial cut plane 78 performed prior to any bone cutting or implantation. The actual implant surgery of step 242 selects and uses actual or physical, i.e., non-simulated, versions of the femoral implant component and the tibial implant component and performs actual or physical cuts for the distal femoral cut plane and the proximal tibial cut plane according to the corresponding simulated versions.

It should be appreciated that some or all of the above procedures may be performed intraoperatively after some procedures have been completed, for example, after balancing of soft tissue.

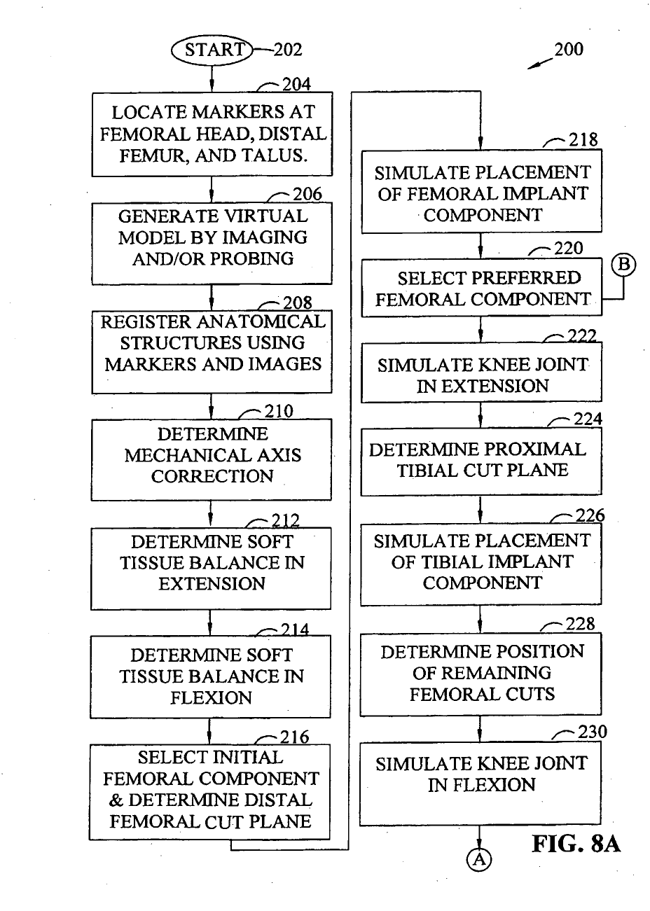

In step 244, computer-assisted surgical system 20 may be utilized to provide guidance in cutting the earlier-determined cut planes. Specifically, as shown in FIG. 7, robotic arm 84 may be used to position cut guide 86 in order to cut actual proximal tibial cut plane 78 and other cut planes using cutting instrument 88. Computer 23 may be preprogrammed with the geometry of cut guide 86 and robotic arm 84 in order to accurately position blade slot 90 and properly locate proximal tibial cut plane 78. Alternatively, other navigational instruments having reference devices 50 may be utilized to provide navigation guidance for locating the earlier-determined cut planes.

In step 246, soft tissue releases or advances are performed to adjust and provide a final soft tissue balance to knee joint 64. In one embodiment, an acceptable level of soft tissue balance in flexion, extension, and during a full range of motion may be a consistent over-tension that may be relieved in step 246. Computer 23 may be used to virtually predict the amount of soft tissue release required to achieve satisfactory soft tissue balance of knee joint 64. Alternatively, step 246 may be completed before the implant placement of step 242. In step 248, method 200 is complete.

While this invention has been described as having exemplary designs, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method for simulating prosthetic implant selection and placement in an anatomical structure using a computer-assisted surgery system, comprising the steps of:
generating a virtual model of the anatomical structure;
registering the anatomical structure with the virtual model of the anatomical structure in the computer-assisted surgery system;
determining a mechanical axis correction of the anatomical structure;
inputting the mechanical axis correction into the computer-assisted surgery system;
determining soft tissue balance in the anatomical structure;
inputting the soft tissue balance into the computer-assisted surgery system;
selecting a simulated implant component corresponding to the mechanical axis correction and the soft tissue balance;
simulating implantation of the simulated implant component;
verifying that the simulated implant component provides the mechanical axis correction and the soft tissue balance; and
selecting an actual implant component corresponding to the simulated implant component if the simulated implant component provides the mechanical axis correction and the soft tissue balance.

2. The method of claim 1, further comprising the additional step of implanting the selected actual implant component in the anatomical structure.

3. The method of claim 2, further comprising the additional step of performing soft tissue releases subsequent to or prior to said implanting step.

4. The method of claim 1, wherein said step of determining a mechanical axis correction comprises manipulating the anatomical structure to form a correct mechanical axis.

5. The method of claim 4, wherein said step of manipulating the anatomical structure comprises moving the anatomical structure through a range of motion and periodically recording a position of the anatomical structure in the computer-assisted surgery system while forming the correct mechanical axis.

6. The method of claim 1, wherein said step of determining soft tissue balance comprises tensioning the anatomical structure to a desired tension.

7. The method of claim 6, wherein said step of tensioning the anatomical structure to a desired tension comprises moving the anatomical structure through a range of motion and periodically recording the desired tension in the computer-assisted surgery system.

8. The method of claim 1, wherein said step of verifying comprises moving the anatomical structure through a range of motion and periodically verifying the simulated implant component provides the mechanical axis correction and the soft tissue balance.

9. A method for simulating prosthetic implant selection and placement in a knee joint using a computer-assisted surgery system, the knee joint including a femur and a tibia, comprising the steps of:
generating a virtual model of the knee joint;
registering the knee joint with the virtual model of the knee joint in the computer-assisted surgery system;
determining a mechanical axis correction of the knee joint;
inputting the mechanical axis correction into the computer-assisted surgery system;
determining soft tissue balance in the knee joint;
inputting the soft tissue balance into the computer-assisted surgery system;
selecting a simulated implant component corresponding to the mechanical axis correction and the soft tissue balance;
simulating implantation of the simulated implant component;
verifying that the simulated implant component provides the mechanical axis correction and the soft tissue balance; and
selecting an actual implant component corresponding to the simulated implant component if the simulated implant component provides the mechanical axis correction and the soft tissue balance.

10. The method of claim 9, further comprising the additional step of implanting the selected actual implant component in the knee joint.

11. The method of claim 10, further comprising the additional step of performing soft tissue releases subsequent to or prior to said implanting step.

12. The method of claim 9, wherein the selected actual implant component comprises one of a distal femoral implant component, a meniscal implant component, and a proximal tibial implant component.

13. The method of claim 9, wherein said step of determining a mechanical axis correction comprises manipulating the knee joint to form a correct mechanical axis.

14. The method of claim 13, wherein said step of manipulating the knee joint comprises moving the knee joint through a range of motion and periodically recording positions of the femur and the tibia in the computer-assisted surgery system while forming the correct mechanical axis.

15. The method of claim 9, wherein said step of determining soft tissue balance comprises tensioning the knee joint to a desired tension.

16. The method of claim 15, wherein said step of tensioning the knee joint comprises moving the knee joint through a range of motion and periodically recording in the computer-assisted surgery system the desired tension between the femur and the tibia.

17. The method of claim 9, wherein said step of verifying comprises simulating movement of the knee joint through a range of motion and periodically verifying the simulated implant component provides the mechanical axis correction and the soft tissue balance.

18. The method of claim 9, further comprising, prior to said simulating step, the additional steps of:
selecting a simulated implant component cut plane corresponding to the mechanical axis correction and the soft tissue balance; and
simulating cutting of the knee joint along the simulated implant component cut plane.

19. The method of claim 18, subsequent to said step of selecting a simulated implant component cut plane, further comprising the additional step of selecting an actual implant component cut plane corresponding to the simulated implant component cut plane if the simulated implant component cut plane provides the mechanical axis correction and the soft tissue balance.

20. The method of claim 19, further comprising the additional step of physically cutting the selected actual implant component cut plane in the knee joint.
